(19) United States
(12) Patent Application Publication
(10) Pub. No.: US 2015/0127049 A1
(43) Pub. Date: May 7, 2015

(54) FILAMENTARY FIXATION DEVICE HAVING PROJECTIONS AND METHOD OF USE

(71) Applicant: HOWMEDICA OSTEONICS CORP., Mahwah, NJ (US)

(72) Inventor: José Raúl Marchand, San Juan, PR (US)

(73) Assignee: HOWMEDICA OSTEONICS CORP., Mahwah, NJ (US)

(21) Appl. No.: 14/073,316

(22) Filed: Nov. 6, 2013

(51) Int. Cl. A61B 17/04

(52) U.S. Cl. CPC A61B 17/0401; A61B 2017/0427; A61B 2017/0445; A61B 2017/0464
USPC 606/232

(57) ABSTRACT

In one embodiment, the present invention includes a fixation device including a cannulated body having a length and a pathway along at least a portion of the length and formed of a filamentary material, the body including at least one projection along its length. The present invention also includes various embodiments of securing such a device within a prepared hole in bone and for securing soft tissue to bone using such a device.

### BACKGROUND OF THE INVENTION

A recent trend in tissue anchor and suture anchor devices is the “soft” device, also referred to as a “filamentary” fixation device, in which the device itself is constructed of a filamentary material, such as suture or the like. Such filamentary fixation devices can replace traditional metal or hard polymer devices in numerous orthopedic surgical procedures, including soft tissue repair and replacement surgical procedures. Such filamentary fixation devices may provide solutions to various problems encountered with traditional metal or hard polymer devices. In many instances, such traditional devices tend to be large in diameter, and must include sufficient material, or other additional structures, to withstand the forces pulling against the device, whether via a suture or directly against the device itself. The size of such devices may limit the possible implantation locations in the body, as sufficient bone mass is required to accommodate the device. Moreover, a large hole must be drilled into the bone to allow for passage of the device through the cortical layer and into the cancellous bone. The larger drill holes may be too invasive resulting in excessive loss of healthy bone, or creation of a large repair site. Such filamentary fixation devices can alleviate many of these problems associated with traditional anchors.

Despite the many benefits these filamentary fixation devices provide, instances of, for example, poor bone quality and the like can increase the chances of lower pullout strength and overall effectiveness of the repair. For example, if the bone is too soft, the bone may not provide sufficient resistance to the device which could result in lower pull-out strength of the device.

Therefore, in light of such issues, there is a need in the art for an improved filamentary fixation device capable of having improved pullout strength in all types of bone, including bone of poor quality.

### BRIEF SUMMARY OF THE INVENTION

Generally, the present invention relates to filamentary fixation devices, kits, methods for manufacture or assembly, and methods of use, for example, in soft tissue repair, particularly where soft tissue must be reattached to bone. In such repairs, the filamentary fixation device can be implanted in a prepared bone hole to secure a length of filament to the bone. The filament can then be used to secure the soft tissue to bone. Further, while the majority of embodiments disclosed herein relate to the use of the filamentary fixation device of the present invention as a “suture anchor” for placement in bone, and to attach, reattach or otherwise secure soft tissue thereto, other uses of the filamentary device are also envisioned.

In one embodiment, the present invention includes a fixation device including a cannulated body having a length and a pathway along at least a portion of the length and formed of a filamentary material, the body including at least one projection along its length.

In one variation, the fixation device may include the at least one projection being positioned on an inner surface of the body such that the projection extends into the pathway. The at least one projection may include two or more projections. Further, the two or more projections can be spaced from one another along the length of the body, circumferentially along the inner surface, or both. Alternatively, where the body includes two or more projections, at least one of the projections can be positioned on the inner surface and at least one of the projections can be positioned on an outer surface of the body such that the at least one projection extends away from the outer surface of the body.

In another variation, the at least one projection may be positioned on an outer surface of the body such that the projection extends away from the outer surface of the body. Further, the at least one projection can include two or more projections, and moreover, the two or more projections can be spaced from one another along the length of the body, circumferentially along the outer surface, or both. Alternatively, the body can include two or more projections, wherein at least one of the projections can be positioned on the outer surface and at least one of the projections can be positioned on an inner surface of the body such that the at least one projection extends into the pathway.

In another embodiment, the present invention includes a fixation device including a body formed of filamentary material having a length and a pathway along a longitudinal axis, an outer surface on an exterior of the body and an inner surface defining the pathway, and an at least one projection extending from the body, and a filament having a length, a portion of the length positioned through the pathway.

In yet another embodiment, the present invention is a method of securing a filamentary fixation device in a bone hole including the steps of inserting the filamentary fixation device into the bone hole, the filamentary fixation device including a body formed of filamentary material having a length and a pathway along at least a portion of the length and along a longitudinal axis, an outer surface on an exterior of the body and an inner surface defining the pathway, and an at least one projection extending from the body, the body being folded on itself, and a filament positioned through the pathway; and tensioning the filament in a direction away from the bone hole to compress the filamentary fixation device within the bone hole such that the at least one projection engages an interior surface of the bone hole, wherein the engagement of the bone hole with the at least one projection restricts movement of the filamentary fixation device in the direction of tension.

In still another embodiment, the present invention includes a method of securing a filament to bone, including the steps of inserting at least a portion of the filament into a hole in the bone, the portion of filament having a filamentary fixation device positioned thereon, the device including a body having a length, a pathway and an at least one projection extending from an outer surface of the body, such that the portion of filament passes through the pathway; and tensioning the filament in a direction away from the bone such that the at least one projection of the device engages the bone and restricts movement of the device and the portion of the filament in the direction of tensioning.

In a further embodiment, the present invention includes a method of securing a filament to bone, including the steps of inserting at least a portion of the filament into a hole in the bone, the portion of filament having a filamentary fixation device positioned thereon, the device including a body having a length, a pathway and an at least one projection being positioned on an inner surface of the body such that the projection extends into the pathway, such that the portion of filament passes through the pathway; and tensioning the filament in a direction away from the bone to secure the device in bone. Furthermore, the at least one projection of the device can engage the filament and restrict movement of the filament through the pathway in at least one direction.

In another embodiment, the present invention includes a method of securing soft tissue to bone, including the steps of engaging the soft tissue with a first end of a filament, the filament having a length between the first end and a second end and having a filamentary fixation device positioned along a portion of the length, the device including a body having a length, a pathway and an at least one projection being positioned on an inner surface of the body such that the projection extends into the pathway, such that the portion of filament passes through the pathway; positioning the device within a hole in bone and securing the device therein; and tensioning the filament from the second end to draw the tissue towards the device, wherein the at least one projection of the device engages the filament and restricts movement of the filament through the pathway in a direction opposite the direction of tensioning.

### BRIEF DESCRIPTION OF THE DRAWINGS

FIGS. 1 and 2 illustrate various views of one embodiment of a filamentary fixation device.

FIG. 3 illustrates a view of the first end 18 of the device of FIGS. 1 and 2.

FIG. 4 illustrates a perspective view of the device of FIGS. 1-3.

FIGS. 5a and 5b illustrate various steps of implanting the device of FIGS. 1-4 in a bone hole in bone.

FIG. 6 illustrates another embodiment of a filamentary fixation device.

FIG. 7 illustrates a further embodiment of a filamentary fixation device.

FIG. 8 illustrates yet another embodiment of a filamentary fixation device.

FIG. 9 illustrates the device of FIG. 8 in a compressed state.

FIG. 10 illustrates a view of a first end 118 of the device of FIGS. 8 and 9.

FIG. 11 illustrates still another embodiment of a filamentary fixation device.

FIG. 12 illustrates another embodiment of a filamentary fixation device.

### DETAILED DESCRIPTION

The filamentary fixation devices, kits, methods for manufacture or assembly and methods of use of the present invention are intended for use in the repair, reattachment, replacement or otherwise securement of tissue, including both hard tissue (i.e., bone or the like) and soft tissue. Soft tissue may be, for example, meniscus, cartilage, capsule, ligaments and tendons, replacement grafts of any of these soft tissues, or the like. While many of the exemplary methods of use disclosed herein are directed towards the use of the filamentary fixation devices and kit as a suture anchor for implantation into a bone hole, other uses are also envisioned. As used herein, “proximal” or “proximally” means closer to or towards an operator, e.g., surgeon, while “distal” or “distally” means further from or away from the operator.

As used herein, the term “filament” or “filamentary” is defined as a suture or other thread-like material. Such filament may be constructed of synthetic material (e.g., PLGA, UHMWPE (ultra high molecular weight polyethylene), polyester, PEEK, Nylon, polypropylene, aramids (for example, Kevlar®-based fibers) or the like, or blends thereof), organic material (silk, animal tendon, or the like or blends thereof), or blends of both one or more organic materials and one or more synthetic materials. Alternatively, filaments may include thin metal wires. While any of these materials may be used, it is preferable, and is disclosed herein, that the various filaments or filamentary aspects of the present invention be constructed out of suture, such as UHMWPE, polyester or blends thereof.

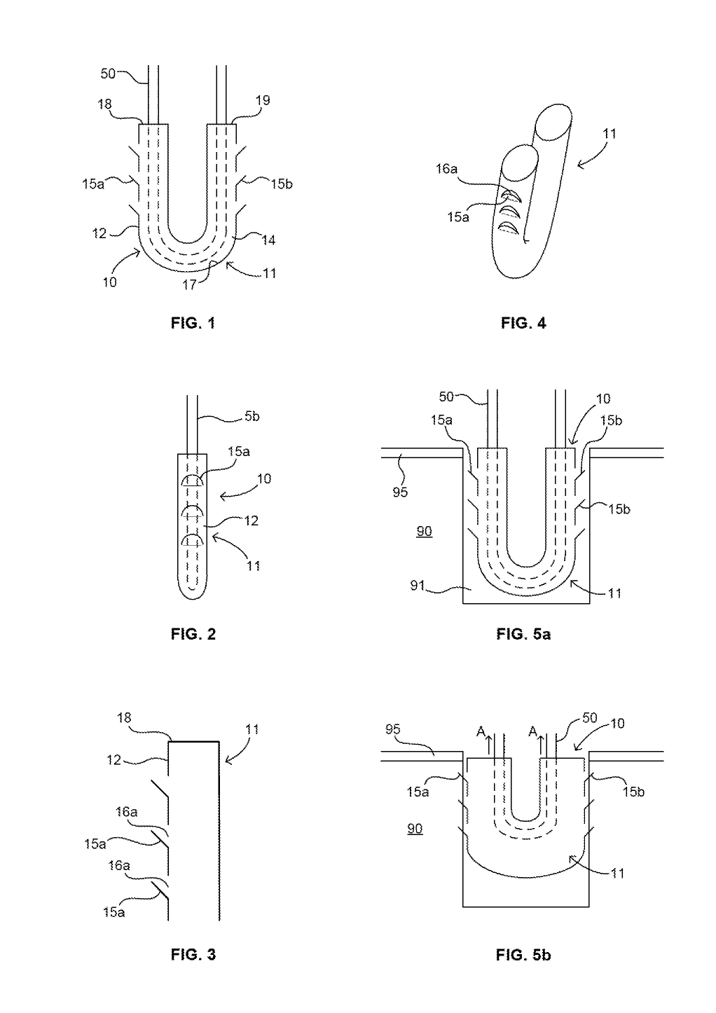

In one embodiment, illustrated in detail in FIGS. 1-5b, a filamentary fixation device, designated as filamentary fixation assembly 10, of the present invention includes a body 11, or sleeve, having a length between a first end 18 and a second end 19 and a cannulation 14, or hollow pathway 14, therethrough, through which at least a portion of a filament 50 can be passed.

The body 11 includes a generally cylindrical shape along a longitudinal axis, defined by the first end 18 and the second end 19, and the hollow pathway 14 extends along the longitudinal axis. The first end 18 and the second end 19 may be perpendicular to the longitudinal axis or they may be at a transverse angle to the longitudinal axis. The device 10 is illustrated with the body 11 folded on itself to form a U-shape, which is the preferred shape for insertion of the device 10 into a bone hole, for example. While this device 10 is one embodiment, it is envisioned that alternative configurations of the device 10 may also be used, and such alternative configurations may include alternative shapes, sizes, or features as desired. Generally speaking, many features of filamentary fixation device 10, and associated instrumentation and implant features, may be similar to those disclosed in U.S. Provisional Application No. 61/679,336, filed Aug. 3, 2012, U.S. application Ser. No. 13/303,849, filed Nov. 23, 2011, Ser. No. 13/588,586, filed Aug. 17, 2012, Ser. No. 13/588,592, filed Aug. 17, 2012, Ser. No. 13/783,804, filed Mar. 4, 2013, and Ser. No. 13/792,982, filed Mar. 11, 2013, and U.S. Pat. Nos. 5,989,252 and 6,511,498, the entireties of which are incorporated by reference herein as if fully set forth herein and all of which are assigned to the same entity as the present application. Another exemplary filamentary fixation device for use in the present invention is the ICONIX™ line of filamentary fixation products (Howmedical Osteonics, Mahwah, N.J.). Other alternative configurations are also envisioned.

Returning to the embodiment of FIGS. 1-5b, the present invention also includes, on the body 11, an at least one projection 15a, 15b, two or more projections, or preferably, a plurality of projections (as illustrated). While device 10 is illustrated as having three projections on each side of the U-shaped body 11, the number of projections may differ, and moreover, the number of projections may differ between the two sides of body 11. Also, as in FIGS. 2 and 4, the projections on a single side may be positioned at the same location circumferentially along the length, or in the alternative each projection may be located at a different location circumferentially, as well as longitudinally along the axis.

The shape of each projection 15a, 15b, as illustrated, is curved, though again, the shape may differ as desired, such that at least one of the projections can be curved, or a different shape, such as chevron-shaped or V-shaped, square-shaped, triangular-shaped, or the like.

Preferably, the shape of the at least one projection 15a, 15b may be a chevron-shape which is prepared by flattening the body 11 and cutting from the edge of the flattened portion to a desired distance through the sleeve at an angle transverse to the longitudinal axis of the body. Alternatively, the curved shape, as illustrated, can be prepared by cutting the outer surface 12 of the body 11 at an angle transverse to the longitudinal axis of the body. Since the body 11 is generally cylindrical in shape, such an angled cut results in a projection having the curved-shape. In one further embodiment, the first end 18 and the second end 19 may be parallel to the transverse angled cuts.

The projections can be formed by any known means. Typically, the projections are formed by cutting the body at a desired location to form the projection. The cut(s) can extend through less than 180 degrees of the circumference of the outer surface of the body, whether the cut is perpendicular to the longitudinal axis of the body or at an angle transverse to this axis. Upon formation of the projection or projections, a hole may be formed through the body 11 from the outer surface 12 to an inner surface 17 and into the cannulation 14. The resulting hole is simply the byproduct of forming the projection as the cut passes completely through the thickness of the wall of the body, defined between the outer surface 12 and the inner surface 17. Such resulting hole may be beneficial in terms of tissue in-growth into the body.

For example, in one embodiment, a hot knife may be used to cut the body 11 along at least a portion of the outer surface 12 along its circumference. The hot knife may also melt or otherwise soften the body material, both the material now forming the projections as well as the adjacent material. Upon completion of the cut, the softened material will cool and may harden to be stiffer and less flexible than prior to the cut, though the hardened portions of material may still have a degree of flexibility. This hardening step can be particularly beneficial to the projections as the hardened projections may impart a stronger locking force upon engagement with bone (discussed below).

Alternatively, an automated process using a blade, laser or the like may be used in place of the hot knife process. In this example, the use of the laser may impart greater precision on the cutting technique, and additionally, the laser can more easily form unique or detailed cutting patterns to form projections having complex shapes.

Whether using the hot knife, laser or other instrument for forming the cuts, the projections may be formed all at once (e.g., by a series of hot knife “blades” which form the cuts all at once along the length of the body, or length of filament which is later cut into distinct, separate bodies) or individually along the length of the body or length of filament.

In still another embodiment of forming the cuts, the entirety of the body 11 (or length of filament later divided into separate bodies) can be heated to a temperature just below the glass transition temperature of the material used. The body may then be allowed to cool. Due to the elevated temperature, upon cooling the body remains flexible but is harder or stiffer than prior to the heating process. The cuts may then be formed using any technique desired. This process may result in a stiffer body, including stiffer projections (as discussed above relative to the hot knife embodiment).

In yet a further embodiment, an adhesive may be used to stiffen a portion of the body 11, such as the projections or the entire body. Such an adhesive would stiffen the material to which it was applied. One example of a suitable adhesive is Histoacryl® (Aesculap, Inc., Center Valley, Pa.). The adhesive may be applied before or after the cuts are made to form the projections.

Furthermore, following cutting the filament, whether to form the separate bodies or to form the projections, heat may be applied to any cut portion of the filament to harden the end portion of the filament and/or projection as well as to prevent fraying at the point of cutting.

In yet another alternative embodiment, the projections may be separate from the body 11 and applied to the body using an adhesive, a weaving step or the like. Such projections may be the same or different material than the filament. In one example, such projections may be a rigid or semi-rigid plastic which are applied to the filament using an adhesive.

Projections 15a, 15b are preferably positioned on the body 11 such that they can contact the surface of the bone hole. As illustrated in FIGS. 5a, 5b, the projections 15a, 15b are positioned towards first and second ends 18, 19 of the body 11 and on an outer side of the body 11 once in the U-shape (as illustrated) such that, once the device 10 is positioned in the bone hole 91, the projections 15a, 15b can engage the surface of the bone hole. Further, the projections are angled upwards such that they are better-positioned to counteract any potential pullout force experienced on the body 11, commonly from tension applied to the filament 50. As will be discussed in greater detail below, a filament 50, positioned through the passageway 14 of body 11, can be tensioned (in the direction of arrow “A”) which compresses the body 11 and forces the projections 15a, 15b against the surface of the bone hole. In this position, the projections may provide additional pull-out strength against tension applied by the filament 50.

For example, as in FIGS. 5a and 5b, as tension is applied to the filament 50, in direction A (i.e., away from the bone and bone hole), the distal-most portion (i.e., the bottom of the “U”) of body 11 is pulled proximally by the filament 50 while the surface of the bone hole 91 counteracts this proximal force through friction against the body 11. The combination of these forces can compress the body 11, as illustrated between FIGS. 5a and 5b. As the body 11 is compressed, the cuts in the outer surface 12 of the body 11 open such that the projections 15a, 15b move outward and towards the surface of the bone hole 91. As the projections move outward, they contact the surface of the bone hole, which can result in even further friction between the body 11 and the bone hole surface. In this position, the filament 50 may then be used to secure tissue against the bone surface 95, or the like. In one embodiment the cuts in the outer surface 12 of the body 11 are positioned proximate to the first end 18 and the second end 19 such that when in the bone, the body is folded into a u-shaped configuration and the cuts are positioned only on a proximal portion of the body (not shown). In this embodiment the projections increase the relative friction on the proximal portion of the body when tension is applied to the filament 50 to improve the compression of the distal-most portion of the body into the proximal portion.

Both the body 11 and the filament 50 are formed of filamentary material, whether the same material or different materials. While the material of the body 11 should be hollow, the filament 50 can either be a hollow filament or a solid filament (i.e., having a core). The filament 50, as it may be positioned through the cannulation, should have a diameter less than or equal to the diameter of the cannulation 14 of the body 11, and as such the body should have an outer diameter that is larger than the diameter of the filament. In one example, the body may be constructed of #5 suture while the filament may be constructed of #1 or #2 filament. In another example, the body may preferably have an outer diameter of about 2.3 mm.

FIG. 6 illustrates another embodiment of an assembly 10 having body 11, labeled assembly 10′ and body 11′. This embodiment is similar to that of FIGS. 1-5b, with the exception that body 10′ includes at least one opening 13a′. While four openings 13a′, 13b′, 13c′, 13d′ are illustrated, body 10′ can include any number of openings desired. The inclusion of such openings on the body of a filamentary fixation device was previously disclosed in U.S. application Ser. No. 13/303,849, incorporated by reference above, as well as in the ICONIX™ line of filamentary fixation products. As described in depth in the '849 Application, such openings can provide for additional benefits including improved compression of the body 11′ within bone. As illustrated, body 11′ can include at least one projection 15a′, 15b′, similar to those described above.

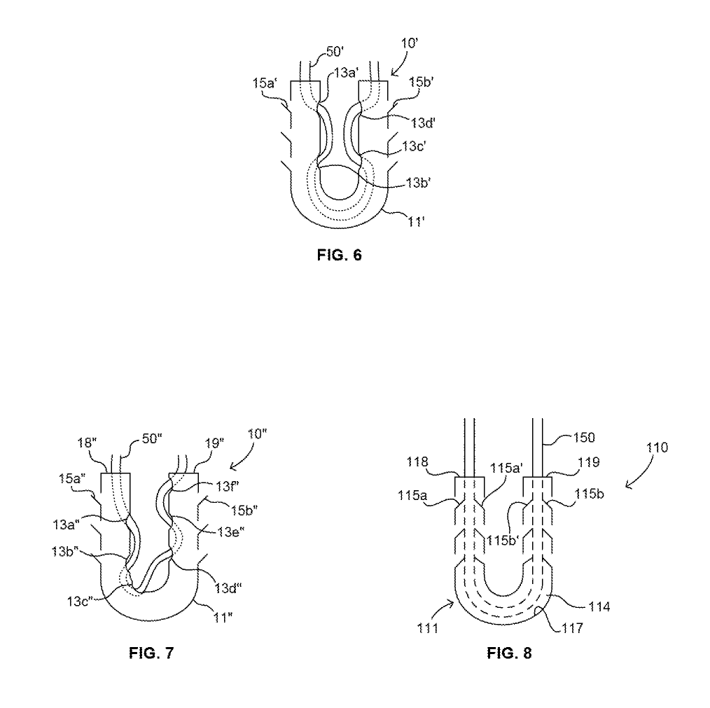

FIG. 7 illustrates another alternative embodiment of an assembly including a body, labeled assembly 10″ and body 11″. In this variation, the openings 13a″, 13b″, 13c″, 13d″, 13e″, 13f″ are staggered relative to the two sides 18″, 19″ such that the filament 50″, upon exiting the body 11″, does so along portions of the body 11″ on one end 18″ staggered from the other end 19″. Such staggering may result in a more compact compression of the body 11″ which may result in even better fixation from increased friction over the compression achieved from body 11, 11′. Such staggering may also allow for insertion of the body into a smaller bone hole, thereby decreasing the trauma to the bone during the surgical procedure.

In another embodiment, illustrated in FIGS. 8-10, the filamentary fixation device 110 has a body 111 including a generally cylindrical shape along a longitudinal axis, defined by a first end 118 and a second end 119, and a hollow pathway 114 extending along the longitudinal axis. The device 110 is illustrated with the body 111 folded on itself to form a U-shape, which is the preferred shape for insertion of the device 110 into a bone hole, for example.

Continuing with FIGS. 8-10, the present invention also includes, on the body 111, an at least one projection 115a, 115b, two or more projections, or preferably, a plurality of projections (as illustrated). As illustrated, in a preferred configuration, the body 111 would include at least one projection 115a, 115a′ on each opposing side of the inner surface 117 on one side of the body 111 towards end 118 and at least one projection 115b, 115b′ on each opposing side of the inner surface 117 on the other side of the body 111 towards end 119. While device 110 is illustrated as having three sets of opposing projections on each side of the U-shaped body 111, the number of projections may differ, and moreover, the number of projections may differ between the two sides of body 111 the opposing sides of each side of the body, and the like. The variations of the size, location, shape, and the like of each projection on body 11 are equally applicable here to body 111.

Moreover, in an even further alternative, the projections need not be only on opposing sides, and thus facing one another, as in FIGS. 8-10, but projections can even be positioned at various locations around the circumference of the inner surface 117. For example, projections can be positioned in a “spiral” or helical pattern around the circumference of the inner surface 117 along a portion of the length of the body 111 extending from one or both of the ends 118, 119.

Such projections may be formed by any of the methods discussed above. Further, the body 111 and filament 150 may be formed of any filamentary material desired, as discussed above.

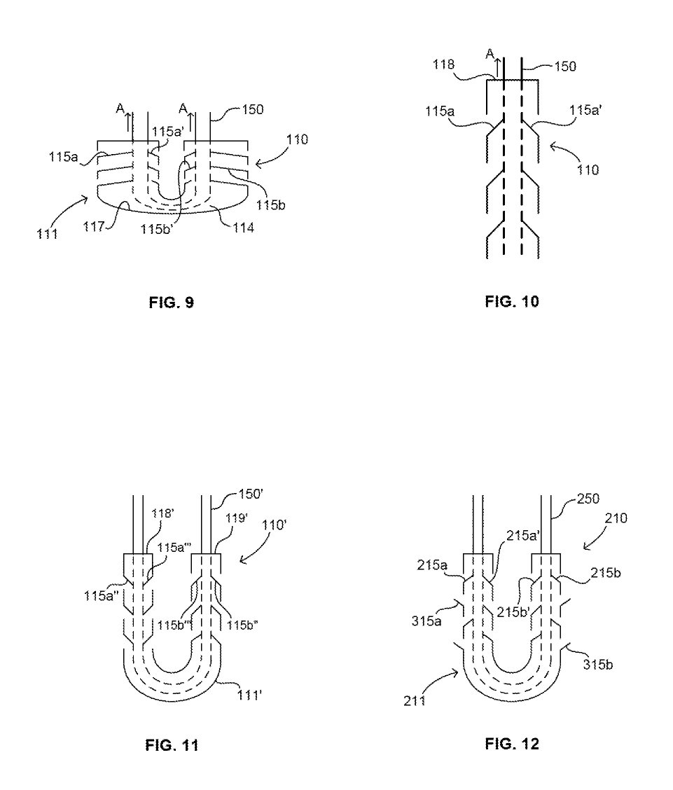

These inner projections 115a, 115a′, 115b, 115b′ may contact the filament 150 when the filament is positioned through the pathway 114 of body 111. As illustrated, the projections may be positioned at an angle transverse to the longitudinal axis of the pathway 114 such as to allow generally one-way travel of the filament 150 through the pathway. In one example, illustrated in FIGS. 8 and 9, as the filament 150 is tensioned in direction “A,” the filament 150 can compress the body 111, and relatively speaking, the filament 150 travels out of and away from the body 111 (i.e., as the filament portion in the distal-most portion of the body 111, the U-turn, is pulled upward, the filament generally moves upwards, or proximally and away from the body). During this movement, the projections may deflect away from the filament, or merely by virtue of their angle of formation, the projections provide little resistance to this movement by the filament. However, once tension is released from the filament 150, the projections serve to prevent the filament from travelling back into the body 111, which would allow the body to expand back to its original shape. As such, the projections form a one-way locking mechanism on the filament such that the filament may be tensioned (either when deploying or compressing the body, or when securing tissue to the bone) but once such tension is relaxed, the projections may prevent the filament from moving back into the body. Such locking of the filament can also prevent the filament from moving axially through the pathway of the body 11, such that the filament is held in place within the pathway.

In one alternative embodiment, illustrated in FIG. 11, apparatus 110′ includes body 111′ and filament 150′, similar to apparatus 110. However, body 111′ includes one difference relative to the projections 115a″, 115a′″, 115b″, 115b′″ in that projections 115a″, 115a′″ are angled in the same direction as projections 115b″, 115b″′ (when viewed with the body 111′ in a linear position). With the projections positioned in this manner, the projections can allow for one-way sliding (or axial movement) of the filament 150′ through the body 111′ (i.e., towards end 119′), but prevent sliding in the other direction (i.e., towards end 118′). Such an arrangement of projections can be useful, for example, when tensioning soft tissue secured to one end (i.e., end of filament extending from end 118′). In this example, after the body 111′ has been deployed (e.g., compressed within the bone by pulling on filament 150′ end extending from end 119′ and holding the end of the filament extending from end 118′), the filament 150′ slides axially through body 111′ and through end 119′. Upon such tensioning, the projections prevent axial sliding of the filament 150′ through the body 111′ in the opposite direction (i.e., towards end 118′) such that the tension on the soft tissue may remain even once the filament 150 (extending from end 119′) is released.

FIG. 12 illustrates yet another embodiment of the present invention. In this embodiment, the device 210 includes a body 211 that includes both outward-facing projections 315a, 315b and inward-facing projections 215a, 215a′, 215b, 215b′. As discussed above relative to both types of projections, each of the projections can have a certain shape, size and angle of projection as desired, and the number of projections at each location can also vary as desired.

This embodiment as illustrated in FIG. 12 provides projections suitable for both engaging the surface of the bone hole 91 (projections 315a, 315b) and those for engaging the filament 250 (projections 215a, 215a′, 215b, 215b′). Thus, with these various projections on body 211, additional friction between the body and both the filament and the surface of the bone hole can further improve pull-out strength of the body. Of course, as another alternative, projections 215a, 215a′, 215b, 215b′ could have the configuration illustrated in FIG. 11, to allow one-way axial sliding of the filament through the body 211.

In another embodiment, the present invention includes a kit comprising at least one filament and a plurality of filamentary fixation devices. The devices may vary in size, number of projections, location of projections, or the like, or alternatively each of the devices in the kit can be identical to each other. Similarly, the filament may be suitable for use in any of the devices, or alternatively the kit can include a plurality of filaments of the same diameter and length or of various diameters and/or lengths. The kit can include the filaments and devices individually, or alternatively, at least one filament can be positioned through at least one device. The kit may further include additional elements such as insertion instruments, drills and/or reamers, or the like. Further, the kit could include a filament loading shuttle or device, for example, such as those described in U.S. Provisional Application No. 61/679,336, filed Aug. 3, 2012 and U.S. application Ser. No. 13/588,586, filed Aug. 17, 2012, Ser. No. 13/588,592, filed Aug. 17, 2012, Ser. No. 13/783,804, filed Mar. 4, 2013, and Ser. No. 13/792,982, filed Mar. 11, 2013, all of which were incorporated by reference above.

The various embodiments of the filamentary fixation devices of FIGS. 1-12 can be used in a variety of methods of use to repair soft tissue, reattach tissue to bone, secure a filament to bone, secure a filamentary fixation device within bone, and the like. While certain exemplary embodiments are discussed below, it is envisioned that the devices and kits of the present invention can be used in other ways than those explicitly described here.

In one embodiment, a method of securing a filamentary fixation device in a bone hole includes the steps of inserting the filamentary fixation device 10, 10′, 10″, 110, 110′, 210 into the bone hole, the device having a filament 50, 50′, 50″, 150, 150′, 250 positioned therethrough, and tensioning the filament in a direction away from the bone hole to compress the filamentary fixation device within the bone hole such that an at least one projection moves outward from the outer surface (represented as 12, 112, 212 in certain figures) of the body 11, 11′, 11″, 111, 111′, 211 and/or inward from the inner surface (represented as 17, 117, 217 in certain figures) of the body.

If the projection is intended to move outward, then the tensioning step would move the projection into engagement with an interior surface of the bone hole 91, wherein the engagement of the bone hole with the projection restricts movement of the filamentary fixation device in the direction of tension. However, if the projection is intended to move inward, then the tensioning step would move the projection into engagement with the filament, wherein the engagement of the projection with the filament restricts movement of the filament, within the body, in at least one direction, even upon release of the tension on the filament. Of course, if the device 210 is used in this method, upon tensioning the filament, at least one projection may engage the surface of the bone hole and at least one projection may engage the filament within the pathway in the body.

Once tension has been applied, and the body 11, 11′, 11″, 111, 111′, 211 has been deployed, the filament may be used to perform a surgical repair of tissue, such as, for example, reattaching soft tissue to bone, for example a rotator cuff, shoulder labrum, hip labrum, or the like. The body having the at least one projection, and preferably a plurality of projections (directed outward and/or inward), may provide additional pullout strength against tension applied to the filament by the soft tissue, or other mass attached thereto. For example, one end of the filament may be secured to the soft tissue to be repaired, and the other end of the filament may be tensioned to tension the soft tissue towards the body and its position in the bone. The filament may then be secured to maintain tension on the soft tissue. In one variation, where the body includes projections on the inside, as with FIG. 11, the projections 115a″, 115a″′, 115b″, 115b′″ may allow for tensioning of the filament, and the soft tissue, and prevent the filament from sliding in the opposite direction through the body, thereby restricting the filament and preventing loss of tension on the soft tissue. While the projections may be sufficient to maintain such tension, the operator may secure the filament in an additional manner (knots, adhesive, suture fastener, or the like) to ensure stability of the repair.

In another embodiment, a method of securing a filament to bone includes the steps of inserting the filamentary fixation device 10, 10′, 10″, 110, 110′, 210 into the bone hole, the device having a filament 50, 50′, 50″, 150, 150′, 250 positioned therethrough, and tensioning the filament in a direction away from the bone hole to compress the filamentary fixation device within the bone hole such that an at least one projection moves outward from the outer surface (represented as 12, 112, 212 in certain figures) of the body 11, 11′, 11″, 111, 111′, 211 or inward from the inner surface (represented as 17, 117, 217 in certain figures) of the body. Upon compression of the device within the bone hole, the filament is secured to the bone via the device. If the at least one projection of the device moves outward, the filament may still remain free to travel longitudinally through the compressed device such that the length of the individual filament ends, extending from the device, can be adjusted. However, if at least one of the projections moves inward, the filament may be prevented from travelling through the pathway in at least one direction. The filament may then be used to secure tissue, or other mass, to the bone.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A fixation device comprising a cannulated body having a length and a pathway along at least a portion of the length and formed of a filamentary material, the body including at least one projection along its length.

2. The device of claim 1, wherein the at least one projection is positioned on an outer surface of the body such that the projection extends away from the outer surface of the body.

3. The device of claim 2, wherein the body includes two or more projections, wherein the two or more projections are positioned on the outer surface.

4. The device of claim 3, wherein the two or more projections are spaced from one another along the length of the body, circumferentially along the outer surface, or both.

5. The device of claim 1, wherein the projection is formed by cutting the body in a direction transverse to a longitudinal axis positioned through the pathway.

6. The device of claim 5, wherein the body includes two or more projections, and each of the projections is substantially the same size as the other projections.

7. The device of claim 5, wherein the projection has a curved shape, a chevron shape, a V-shape, a triangular shape or a square shape.

8. The device of claim 5, wherein upon forming the projections, the body has a hole extending from an outer surface and into the pathway.

9. The device of claim 1, wherein a portion of a filament is positioned through the pathway.

10. The device of claim 9, wherein the device includes at least one opening through which the filament can pass through the device from the pathway to a position outside the device.

11. The device of claim 10, wherein the at least one opening includes at least one opening adjacent a first end of the device and at least one opening adjacent a second end of the device, wherein upon folding the device on itself, the openings are symmetrically positioned adjacent each end of the device.

12. The device of claim 10, wherein the at least one opening includes at least one opening adjacent a first end of the device and at least one opening adjacent a second end of the device, wherein upon folding the device on itself, the openings are offset relative one another.

13. A fixation device to be secured in a bone hole in bone, comprising:
a body formed of filamentary material having a length and a pathway along a longitudinal axis, an outer surface on an exterior of the body and an inner surface defining the pathway, and an at least one projection extending from the body, and
a filament having a length, a portion of the length positioned through the pathway.

14. The fixation device of claim 13, wherein the projection is positioned on the outer surface and extends away from the body.

15. The fixation device of claim 13, wherein the projection extends from the body at an acute angle relative to the longitudinal axis.

16. The fixation device of claim 13, wherein the projection is formed by a circumferential cut through the body extending from the outer surface and through the inner surface.

17. The fixation device of claim 16, wherein the cut extends through a portion of the circumference of the body.

18. The fixation device of claim 16, wherein the cut extends through less than 180 degrees of the circumference of the body.

19. A method of securing a filament to bone, comprising:
inserting at least a portion of the filament into a hole in the bone, the portion of filament having a filamentary fixation device positioned thereon, the device including a body having a length, a pathway and an at least one projection extending from an outer surface of the body, such that the portion of filament passes through the pathway; and
tensioning the filament in a direction away from the bone such that the at least one projection of the device engages the bone and restricts movement of the device and the portion of the filament in the direction of tensioning.

20. The method of claim 19, wherein the tensioning of the filament compresses the filamentary fixation device within the bone hole such that the at least one projection moves outward from the device and towards the bone.
